(19) United States
(12) Patent Application Publication
(10) Pub. No.: US 2011/0160849 A1
(43) Pub. Date: Jun. 30, 2011

(54) BIMODAL TRICUSPID ANNULOPLASTY RING

(75) Inventors: Alain F. Carpentier, Paris (FR); Alison S. Curtis, Irvine, CA (US)

(73) Assignee: Edwards Lifesciences Corporation, Irvine, CA (US)

(21) Appl. No.: 12/975,092

(22) Filed: Dec. 21, 2010

Related U.S. Application Data
(60) Provisional application No. 61/289,238, filed on Dec. 22, 2009.

(51) Int. Cl. A61F 2/24

(52) U.S. Cl. 623/2.19; 623/2.36

(57) ABSTRACT

A prosthetic remodeling tricuspid annuloplasty ring having two free ends can be configured to more accurately mimic native valve anatomy (e.g., shape) and movement during the cardiac cycle. A tricuspid ring can be provided with a substantially elliptical shape in the X-Y plane, and a bimodal saddle shape in the Z direction. The tricuspid ring can be configured to contract and expand during each cardiac cycle such that the area of the orifice and/or the diameter of the ring decrease with each contraction. Further, the elevation or non-planarity of the bimodal saddle shape can increase with each contraction. Movement of the tricuspid ring can vary in each different segment of the tricuspid ring. Tricuspid annuloplasty rings can be provided in a set, with changing ratios of diameter, changing out-of-plane static amplitudes, and changing amounts of dynamic movement in each different size of tricuspid ring.

### RELATED APPLICATIONS

The present application claims priority under 35 U.S.C. §119 to U.S. Provisional Application No. 61/289,238, filed on Dec. 22, 2009, which is incorporated herein by reference in its entirety.

### FIELD OF THE INVENTION

The present invention relates generally to medical devices and particularly to a tricuspid annuloplasty ring.

### BACKGROUND OF THE INVENTION

In vertebrate animals, the heart is a hollow muscular organ having four pumping chambers: the left and right atria and the left and right ventricles, each provided with its own one-way valve. The native heart valves are identified as the aortic, mitral (or bicuspid), tricuspid, and pulmonary, and each is mounted in an annulus comprising dense fibrous rings attached either directly or indirectly to the atrial and ventricular muscle fibers. Each annulus defines a flow orifice.

Heart valve disease is a widespread condition in which one or more of the valves of the heart fails to function properly. Diseased heart valves may be categorized as either stenotic, wherein the valve does not open sufficiently to allow adequate forward flow of blood through the valve, and/or incompetent, wherein the valve does not close completely, causing excessive backward flow of blood through the valve when the valve is closed (regurgitation). Valve disease can be severely debilitating and even fatal if left untreated.

A healthy tricuspid valve annulus is substantially ovoid in the X-Y plane, having a bimodal saddle shape in the Z direction. A diseased tricuspid valve annulus is often substantially flat in the Z direction, and can experience severe distension in the X-Y plane. During the cardiac cycle, a healthy valve annulus typically expands in the X-Y direction, as well as slightly accentuates the saddle in the Z direction. In diseased valves, there is often suppressed orifice expansion, as well as substantially no saddle accentuation during the cardiac cycle.

Various surgical techniques may be used to repair a diseased or damaged valve. In a valve replacement operation, the damaged leaflets are excised and the annulus sculpted to receive a replacement valve. Another less drastic method for treating defective valves is through repair or reconstruction, which is typically used on minimally calcified valves. One repair technique is remodeling annuloplasty, in which the deformed valve annulus is reshaped by attaching a prosthetic annuloplasty repair segment or ring to the valve annulus. The annuloplasty ring is designed to support the functional changes that occur during the cardiac cycle: maintaining coaptation and valve integrity to prevent reverse flow while permitting good hemodynamics during forward flow.

An annuloplasty ring typically comprises an inner substrate of a metal such as rods or bands of stainless steel or titanium, or a flexible material such as silicone rubber or Dacron cordage, covered with a biocompatible fabric or cloth to allow the ring to be sutured to the fibrous annulus tissue. Annuloplasty rings may be stiff or flexible, split or continuous, and may have a variety of shapes, including circular, D-shaped, C-shaped, or kidney-shaped. Examples are seen in U.S. Pat. Nos. 5,041,130, 5,104,407, 5,201,880, 5,258,021, 5,607,471, 6,187,040, and 6,908,482.

FIG. 1 shows a schematic representation of the anatomic orientation of the heart, illustrating the atrioventricular (AV) junctions within the heart and the body in the left anterior oblique projection. The body is viewed in the upright position and has three orthogonal axes: superior-inferior, posterior-anterior, and right-left.

FIG. 2 is a cutaway view of the heart from the front, or anterior, perspective, with most of the primary structures marked. As is well known, the pathway of blood in the heart is from the right atrium to the right ventricle through the tricuspid valve, to and from the lungs, and from the left atrium to the left ventricle through the mitral valve. The present application has particular relevance to the repair of the tricuspid valve, which regulates blood flow between the right atrium and right ventricle, although certain aspects may apply to repair of other of the heart valves. The tricuspid and mitral valves together define the AV junctions.

As seen in FIG. 2, four structures embedded in the wall of the heart conduct impulses through the cardiac muscle to cause first the atria then the ventricles to contract. These structures are the sinoatrial node (SA node), the atrioventricular node (AV node), the bundle of His, and the Purkinje fibers. On the rear wall of the right atrium is a barely visible knot of tissue known as the sinoatrial, or SA node. This tiny area is the control of the heart's pacemaker mechanism. Impulse conduction normally starts in the SA node. It generates a brief electrical impulse of low intensity approximately 72 times every minute in a resting adult. From this point, the impulse spreads out over the sheets of tissue that make up the two atria, exciting the muscle fibers as it does so. This causes contraction of the two atria and thereby thrusts the blood into the empty ventricles. The impulse quickly reaches another small, specialized knot of tissue known as the AV node, located between the atria and the ventricles. This node delays the impulse for about 0.07 seconds, which is exactly enough time to allow the atria to complete their contractions. When the impulses reach the AV node, they are relayed by way of the several bundles of His and Purkinje fibers to the ventricles, causing them to contract. As those of skill in the art are aware, the integrity and proper functioning of the conductive system of the heart is critical for good health.

FIG. 3 is a schematic view of the tricuspid valve orifice seen from its inflow side (from the right atrium), with the peripheral landmarks labeled as: antero-septal commissure, anterior leaflet, posterior commissure, posterior leaflet, postero-septal commissure, and septal leaflet. Contrary to traditional orientation nomenclature, the tricuspid valve is nearly vertical, as reflected by these sector markings. From the same viewpoint, the tricuspid valve is shown surgically exposed in FIG. 4 with an annulus 22 and three leaflets 24a, 24b, 24c extending inward into the flow orifice. Chordae tendineae 26 connect the leaflets to papillary muscles located in the right ventricle to control the movement of the leaflets. The tricuspid annulus 22 is an ovoid-shaped fibrous ring at the base of the valve that is less prominent than the mitral annulus, but larger in circumference.

Reflecting their true anatomic location, the three leaflets in FIG. 4 are identified as septal 24a, anterior 24b, and posterior (or “mural”) 24c. The leaflets join together over three prominent zones of apposition, and the peripheral intersections of these zones are usually described as commissures 28. The leaflets 24 are tethered at the commissures 28 by the fan-shaped chordae tendineae 26 arising from prominent papillary muscles originating in the right ventricle. The septal leaflet 24a is the site of attachment to the fibrous trigone, the fibrous “skeletal” structure within the heart. The anterior leaflet 24b, the largest of the 3 leaflets, often has notches. The posterior leaflet 24c, the smallest of the 3 leaflets, usually is scalloped.

The ostium 30 of the right coronary sinus opens into the right atrium, and the tendon of Todaro 32 extends adjacent thereto. The AV node 34 and the beginning of the bundle of His 36 are located in the supero-septal region of the tricuspid valve circumference. The AV node 34 is situated directly on the right atrial side of the central fibrous body in the muscular portion of the AV septum, just superior and anterior to the ostium 30 of the coronary sinus 30. Measuring approximately 1.0 mm×3.0 mm×6.0 mm, the node is flat and generally oval shaped. The AV node is located at the apex of the triangle of Koch 38, which is formed by the tricuspid annulus 22, the ostium 30 of the coronary sinus, and the tendon of Todaro 32. The AV node 34 continues on to the bundle of His 36, typically via a course inferior to the commissure 28 between the septal 24a and anterior 24b leaflets of the tricuspid valve; however, the precise course of the bundle of His 36 in the vicinity of the tricuspid valve may vary. Moreover, the location of the bundle of His 36 may not be readily apparent from a resected view of the right atrium because it lies beneath the annulus tissue.

The triangle of Koch 30 and tendon of Todaro 32 provide anatomic landmarks during tricuspid valve repair procedures. A major factor to consider during surgery is the proximity of the conduction system (AV node 34 and bundle of His 36) to the septal leaflet 24a. Of course, surgeons must avoid placing sutures too close to or within the AV node 34. C-shaped rings are good choices for tricuspid valve repairs because they allow surgeons to position the break in the ring adjacent the AV node 34, thus avoiding the need for suturing at that location.

One prior art rigid C-shaped ring is the Carpentier-Edwards Classic® Tricuspid Annuloplasty Ring sold by Edwards Lifesciences Corporation of Irvine, Calif., which is seen in FIGS. 5A and 5B. Although not shown, the planar ring 40 has an inner titanium core covered by a layer of silicone and fabric. Rings for sizes 26 mm through 36 mm in 2 mm increments have outside diameters (OD) between 31.2-41.2 mm, and inside diameters (ID) between 24.3-34.3 mm. These diameters are taken along the “diametric” line spanning the greatest length across the ring because that is the conventional sizing parameter. A gap G between free ends 42a, 42b in each provides the discontinuity to avoid attachment over the AV node 34. The gap G for the various sizes ranges between about 5-8 mm, or between about 19%-22% of the labeled ring size. The “ring size” is the size labeled on the annuloplasty ring packaging. As seen in the implanted view of FIG. 6, the gap G is sized just larger than the AV node 34. Despite this clearance, some surgeons are uncomfortable passing sutures so close to the conductive AV node 34, particularly considering the additional concern of the bundle of His 36.

A flexible C-shaped tricuspid ring is sold under the name Sovering™ by Sorin Biomedica Cardio S.p.A. of Via Crescentino, Italy. The Sovering™ is made with a radiopaque silicone core covered with a knitted polyester (PET) fabric so as to be totally flexible. Rings for sizes 28 mm through 36 mm in 2 mm increments have outside diameters (OD) between 33.8-41.8 mm, and inside diameters (ID) between 27.8-35.8 mm. As with other tricuspid rings, a gap between the free ends provides a discontinuity to avoid attachment over the AV node. The gap for the various sizes ranges of the Sovering™ ranges between about 18-24 mm, or between about 60%-70% of the labeled size. Although this gap helps avoid passing sutures close to the conductive AV node 34 and bundle of His 36, the ring is designed to be attached at the commissures on either side of the septal leaflet and thus no support is provided on the septal side.

Whether totally flexible, rigid, or semi-rigid, annuloplasty rings have sometimes been associated with a certain degree of arrhythmia. Prior art annuloplasty rings have also been associated with a 10% to 15% incidence of ring dehiscence and/or conduction tissue disturbance at 10 years post implantation. Additionally, prior art annuloplasty rings have been associated with residual tricuspid regurgitation after implantation. Thus, despite numerous designs presently available or proposed in the past, there is a need for an improved prosthetic tricuspid ring that addresses these and other issues with prior art tricuspid rings.

### SUMMARY OF THE INVENTION

Disclosed embodiments of a tricuspid ring can at least partially restore the correct anatomy of the tricuspid valve annulus and the right ventricle. Tricuspid annuloplasty rings according to the present disclosure can be configured to restore the anatomically correct shape of the valve annulus and right ventricle in all three dimensions and/or to restore the anatomically correct movement of the tricuspid valve. Disclosed tricuspid rings can be combined with a subvalvular apparatus in some embodiments. While the term “tricuspid ring” is used throughout this disclosure, embodiments include both continuous, complete rings and discontinuous rings, with two free ends separated by a gap. Disclosed tricuspid rings are sometimes referred to as having one or more different segments, such as a septal-anterior segment, a lateral-posterior segment, a posterior-septal segment, and an anterior-lateral segment. These segments can correspond to portions of native valve anatomy when the ring is implanted in the valve, as will be described further.

The term “Z axis” in reference to the illustrated rings, and other non-circular or non-planar rings, refers to a line generally perpendicular to the ring that passes through the approximate area centroid of the ring when viewed in plan view. “Axial” or the direction of the “Z axis” can also be viewed as being parallel to the direction of blood flow through the valve orifice, and thus within the ring when implanted therein. Stated another way, the implanted tricuspid ring orients about a central flow axis aligned along an average direction of blood flow through the tricuspid annulus. A “plane” or “X-Y plane” of the ring is perpendicular to the Z axis. However, rings of the present invention are 3-dimensional, meaning that in addition to familiar contours in the X-Y “plane” that can be seen in plan view as looking along the blood flow axis, they also curve up or down from that plane along the flow or Z-axis, as will be seen.

For example, one embodiment of a tricuspid annuloplasty ring for use in a tricuspid valve repair, the tricuspid annulus having peripheral landmarks as viewed from above in a clockwise direction of an antero-septal commissure, anterior leaflet, posterior commissure, posterior leaflet, postero-septal commissure, and septal leaflet, comprising a core made of a relatively rigid material, defined by a septal-anterior segment located around portions of the septal and anterior leaflets when implanted having a free first end and a second end, an anterior-lateral segment located around portions of the anterior and posterior leaflets when implanted having a second end and a first end adjacent the second end of the septal-anterior segment, a lateral-posterior segment located around the posterior leaflet when implanted having a second end and a first end adjacent the second end of the anterior-lateral segment, and a posterior-septal segment located around the septal leaflet when implanted having a free second end and a first end adjacent the second end of the lateral-posterior segment. The tricuspid ring can be configured such that a gap exists between the free first end of the septal-anterior segment and the free second end of the posterior-septal segment. The tricuspid ring can have a bimodal saddle shape having a first and second high point and a first and second low point, the first high point being located within the septal-anterior segment, the second high point being located within the lateral-posterior segment, the first low point being located within the anterior-lateral segment, and the second low point being located within the posterior-septal segment.

In some embodiments, the ratio of the greatest length between any two points on an interior surface of the tricuspid ring to the greatest width between any two points on the interior of the tricuspid ring is at least 1.56. The tricuspid annuloplasty ring can further comprise a subvalvular apparatus. Preferably, the ring is configured to substantially restore the anatomically correct shape in all three dimensions of a native tricuspid valve in which the ring is designed to be implanted. Further, when the ring is positioned within a native tricuspid valve, the first high point of the ring is approximately positioned adjacent the septal-anterior commissure of the native tricuspid valve and the second high point of the ring is approximately positioned adjacent the center of the posterior leaflet of the native tricuspid valve. The elevation of the first high point can be from about 0.5 mm to about 4 mm, and the elevation of the second high point can be from about 2 mm to about 4 mm. The first low point of the ring is approximately positioned adjacent the center of the anterior leaflet of the native tricuspid valve and the second low point of the ring is approximately positioned adjacent the center of the septal leaflet of the native tricuspid valve. The elevation of the first low point is from about −2 mm to about −4 mm. The elevation of the second low point is from about −1 mm to about −4 mm.

The tricuspid annuloplasty ring is configured to move during the normal cardiac cycle once implanted in a native tricuspid valve, such that a first elevation of one or more of the high points and a second elevation of one or more of the low points change during each cardiac cycle. Further, the diameter of the ring can change during each cardiac cycle. The area of the orifice defined by the ring can also change during each cardiac cycle.

In another embodiment of a tricuspid annuloplasty ring for use in a tricuspid valve repair procedure, the tricuspid annulus having peripheral landmarks as viewed from above in a clockwise direction of an antero-septal commissure, anterior leaflet, posterior commissure, posterior leaflet, postero-septal commissure, and septal leaflet, comprising a core made of a relatively rigid material, defined by a septal-anterior segment located around portions of the septal and anterior leaflets when implanted having a free first end and a second end, an anterior-lateral segment located around portions of the anterior and posterior leaflets when implanted having a second end and a first end adjacent the second end of the septal-anterior segment, a lateral-posterior segment located around the posterior leaflet when implanted having a second end and a first end adjacent the second end of the anterior-lateral segment, and a posterior-septal segment located around the septal leaflet when implanted having a free second end and a first end adjacent the second end of the lateral-posterior segment. The ring can be configured such that a gap exists between the free first end of the septal-anterior segment and the free second end of the posterior-septal segment. The ring can have an undulating contour with a local high point located within the septal-anterior segment at the antero-septal commissure when implanted, and a local low point located within the posterior-septal segment. The elevation of the local high point can be from about 0.5 mm to about 4 mm. The tricuspid annuloplasty ring can include a second local high point located within the lateral-posterior segment and having an elevation of from about 2 mm to about 4 mm. The elevation of the local low point is from about −2 mm to about −4 mm. The tricuspid annuloplasty ring can include a second local low point located within the posterior-septal segment and having an elevation of from about −1 mm to about −4 mm.

The ratio of the greatest length between any two points on an interior surface of the tricuspid ring to the greatest width between any two points on the interior of the tricuspid ring can be used to characterize the tricuspid annuloplasty rings disclosed herein. The ratio of the major to minor axis dimensions can be greater than the ratios of conventional tricuspid rings. For example, the ratio can be at least 1.56. Further, the ratio can be altered from one size of tricuspid ring to another. For example, the ratio can decrease as the tricuspid ring size increases. Further, the change in ratio from one size to another size can also change, such that there is a greater change in ratio between larger sizes of tricuspid rings than the change between the ratios of the small sizes of tricuspid rings.

Disclosed embodiments of a tricuspid ring can be three dimensional in shape (e.g., not flat in the Z direction). In some embodiments, a tricuspid ring can be shaped to have a sinusoidal bimodal saddle shape in the Z direction. The amplitude of the sinusoid can be adjustable and can increase with increasing orifice size (e.g., from one size of tricuspid ring to the next). A tricuspid ring can have two high points, and two low points along the Z axis. The high points and low points can be located along different segments of a tricuspid ring. For example, the septal-anterior segment and the lateral-posterior segment can be shaped to form high points of the tricuspid ring, while the posterior-septal segment and the anterior-lateral segment can be shaped to form low points of the tricuspid ring. In some embodiments, the high point of the lateral-posterior segment is higher than the high point of the septal-anterior segment (e.g. has a greater positive displacement along the Z axis). In some embodiments, the low point of the posterior-septal segment is lower than the low point of the anterior-lateral segment (e.g., has a greater negative displacement along the Z axis). In some embodiments, the high point of the septal-anterior segment can be from about 0.5 to about 6 mm in the Z direction (e.g., 0.5 to 6 mm above the X-Y plane at the zero point along the Z axis, or having an elevation of 0.5 to 6 mm), the high point of the lateral-posterior segment can be from about 2 mm to about 6 mm in the Z direction, the low point of the posterior-septal segment can be from about 1 mm to about 6 mm in the negative Z direction (e.g., 1 to 6 mm below the X-Y plane at the zero point along the Z axis), and the low point of the anterior-lateral segment can be from about 2 mm to about 6 mm in the negative Z direction (e.g., the elevation can be from about −2 mm to about −6 mm).

In some embodiments, when the tricuspid ring is implanted in a native tricuspid valve, the first high point of the tricuspid ring can be approximately positioned adjacent the antero-septal commissure of the native tricuspid valve and the second high point of the tricuspid ring can be approximately positioned adjacent the center of the posterior leaflet of the native tricuspid valve. In some embodiments, when the tricuspid ring is positioned within a native tricuspid valve, the first low point of the tricuspid ring can be approximately positioned adjacent the center of the anterior leaflet of the native tricuspid valve and the second low point of the tricuspid ring can be approximately positioned adjacent the center of the septal leaflet of the native tricuspid valve.

Tricuspid rings according to the present disclosure can also be configured to exhibit movement during the normal cardiac cycle after implantation in a native valve. Embodiments of a tricuspid ring can exhibit movement in the X-Y plane and/or in the Z direction during each cardiac cycle. For example, the area of the orifice can expand and contract during the cardiac cycle, such as by expanding by between about 20% and about 40% of its original area. In one embodiment, the area of the orifice can expand by about 29% during each cardiac cycle. In some embodiments, the diameter of the tricuspid ring can expand and contract during the cardiac cycle. For example, the diameter can expand by between about 14.7% and about 17.2% of its static diameter in some embodiments. In one embodiment, the diameter of the tricuspid ring can expand by about 16% during each cardiac cycle.

Disclosed tricuspid rings can also exhibit movement in the Z direction during cardiac cycles after implantation in a valve annulus. For example, a tricuspid ring can undergo sinusoidal bimodal movement in the Z axis, such as by increasing the displacement from the zero point of the Z axis of the high points and low points of the tricuspid ring. In some embodiments, this change in amplitude can increase with increasing ring size (e.g., increasing orifice size). For example, during contraction of the right side of the heart, the amplitude of the bimodal saddle shape can increase in the Z axis, while the area of the orifice and/or the diameter of the tricuspid ring contract. In some embodiments, the changes in displacement from the zero point of the Z axis during contraction can vary by segment. For example, the high point of the septal-anterior segment can move in either direction by about 1 mm, the high point of the lateral-posterior segment can move in either direction by about 1 mm, the low point of the posterior-septal segment can move in either direction by about 1 mm, and the low point of the anterior-lateral segment may not move significantly in some embodiments. In some embodiments, the change in amplitude of the lateral-posterior segment is greater than the change in amplitude of the septal-anterior segment.

Also disclosed is a set of a plurality tricuspid annuloplasty rings. Each tricuspid ring is adapted for use in a tricuspid valve repair procedure, wherein the tricuspid annulus has peripheral landmarks as viewed from above in a clockwise direction of an antero-septal commissure, anterior leaflet, posterior commissure, posterior leaflet, postero-septal commissure, and septal leaflet. Each ring comprises a core made of a relatively rigid material, and is defined by a septal-anterior segment located around portions of the septal and anterior leaflets when implanted having a free first end and a second end, an anterior-lateral segment located around portions of the anterior and posterior leaflets when implanted having a second end and a first end adjacent the second end of the septal-anterior segment, a lateral-posterior segment located around the posterior leaflet when implanted having a second end and a first end adjacent the second end of the anterior-lateral segment, and a posterior-septal segment located around the septal leaflet when implanted having a free second end and a first end adjacent the second end of the lateral-posterior segment. The tricuspid ring can be configured such that a gap exists between the free first end of the septal-anterior segment and the free second end of the posterior-septal segment. The tricuspid ring can have a bimodal saddle shape having a first and second high point and a first and second low point, the first high point being located within the septal-anterior segment, the second high point being located within the lateral-posterior segment, the first low point being located within the anterior-lateral segment, and the second low point being located within the posterior-septal segment. Each tricuspid annuloplasty ring in the set can be partially defined by a ring ratio of the greatest length between any two points on an interior surface of the ring to the greatest width between any two points on the interior of the ring, and the ratio can be different for each tricuspid ring in the set.

The set of tricuspid annuloplasty rings can be ordered from the smallest ring to the largest ring, and the change in the ring ratio from one ring to the next largest ring can be non-constant. In some embodiments, the static elevation of the first and second high points (e.g., the distance of each high point from the X-Y plane bisecting the ring while the ring is static, or at rest) varies with each different sized ring in the set. Further, each tricuspid annuloplasty ring in the set can be configured to move during the normal cardiac cycle when implanted in a native valve such that the elevation of the first and second high points changes during each cardiac cycle. Each tricuspid ring can be configured to undergo a larger change in the elevation of the first and second high points than the next smaller ring in the set.

The elevation of the first and second low points can vary with each different sized ring in the set. Each ring in the set can be configured to move during the normal cardiac cycle when implanted in a native tricuspid valve such that the elevation of the first and second low points changes during each cardiac cycle. Each ring in the set can be configured to undergo a larger change in the elevation of the first and second low points than the next smaller tricuspid ring in the set.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic representation of the AV junctions within the heart and the body in the left anterior oblique projection.

FIG. 2 is a cutaway view of the heart from the front, or anterior, perspective.

FIG. 3 is a schematic plan view of the tricuspid annulus with typical orientation directions noted as seen from the inflow side.

FIG. 4 is a plan view of the native tricuspid valve and surrounding anatomy from the inflow side.

FIGS. 5A and 5B are plan and septal elevational views, respectively, of a planar tricuspid annuloplasty ring of the prior art.

FIG. 6 is a plan view of the native tricuspid valve and surrounding anatomy from the inflow side with the annuloplasty ring of FIGS. 5A-5B implanted.

FIG. 7 is a plan view of one embodiment of a tricuspid ring according to the present disclosure.

FIG. 8 is a perspective view of one embodiment of a tricuspid ring according to the present disclosure.

FIG. 9 is a plan view of a tricuspid valve, with orientation reference points indicated.

FIG. 10 is a plan view of the tricuspid ring according to the present disclosure as in FIG. 7, with segments and saddle points corresponding to FIG. 8.

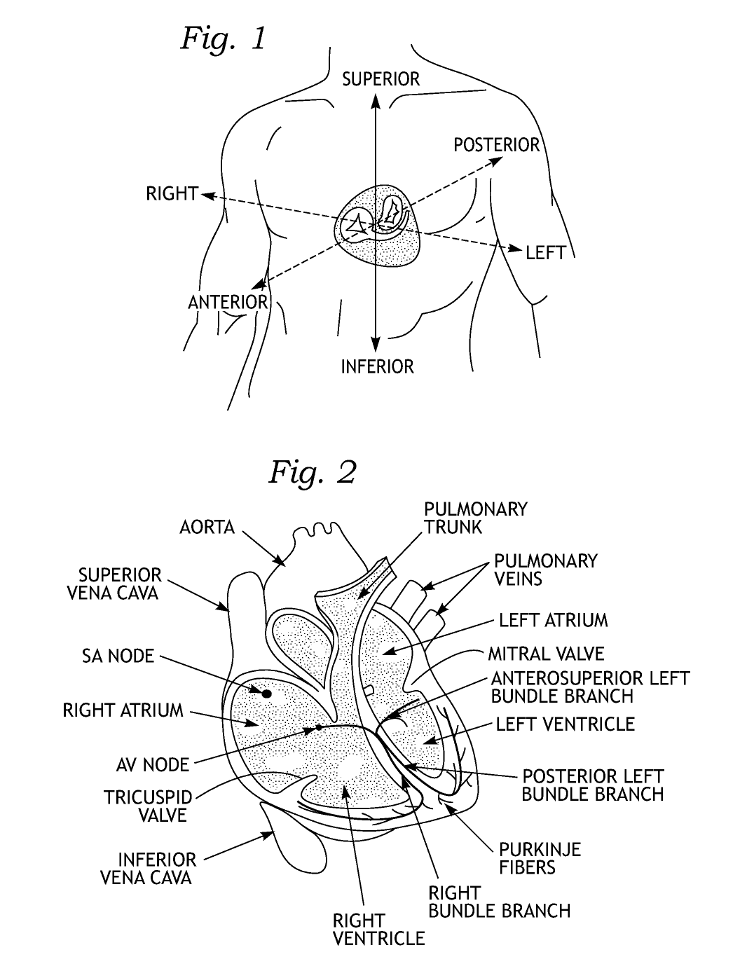

### DETAILED DESCRIPTION OF THE PREFERRED EMBODIMENTS

Embodiments of a tricuspid ring according to the present disclosure can mimic the shape of the native tricuspid valve and right ventricle in order to substantially restore a diseased or damaged annulus to its correct anatomical shape. Tricuspid annuloplasty rings that better conform to the native annulus can be shaped to protect certain features of the surrounding anatomy. The rings of the present disclosure can be designed to support a majority of the tricuspid annulus without risking injury to the leaflet tissue and/or the heart's conductive system, such as the AV node 34 and bundle of His 36 (see FIG. 4). Additionally, disclosed embodiments of a tricuspid ring can be contoured to better approximate the three-dimensional shape of the tricuspid annulus, and can thereby reduce residual tricuspid regurgitation post-operatively. Disclosed embodiments of a tricuspid ring can provide remodeling of diseased tricuspid valve annuluses in a bimodal, anatomically correct shape (e.g., in all three dimensions). Thus, some embodiments can improve durability of the repair by imparting less stress on the native valve leaflets and annulus.

The term “axis” in reference to the illustrated ring, and other non-circular or non-planar rings, refers to a line that passes through the area centroid of the ring when viewed in plan view. “Axial” or the direction of the “axis” can also be viewed as being parallel to the direction of blood flow within the valve orifice and thus within the ring when implanted therein. Stated another way, the implanted tricuspid ring orients about a central flow axis aligned along an average direction of blood flow through the tricuspid annulus.

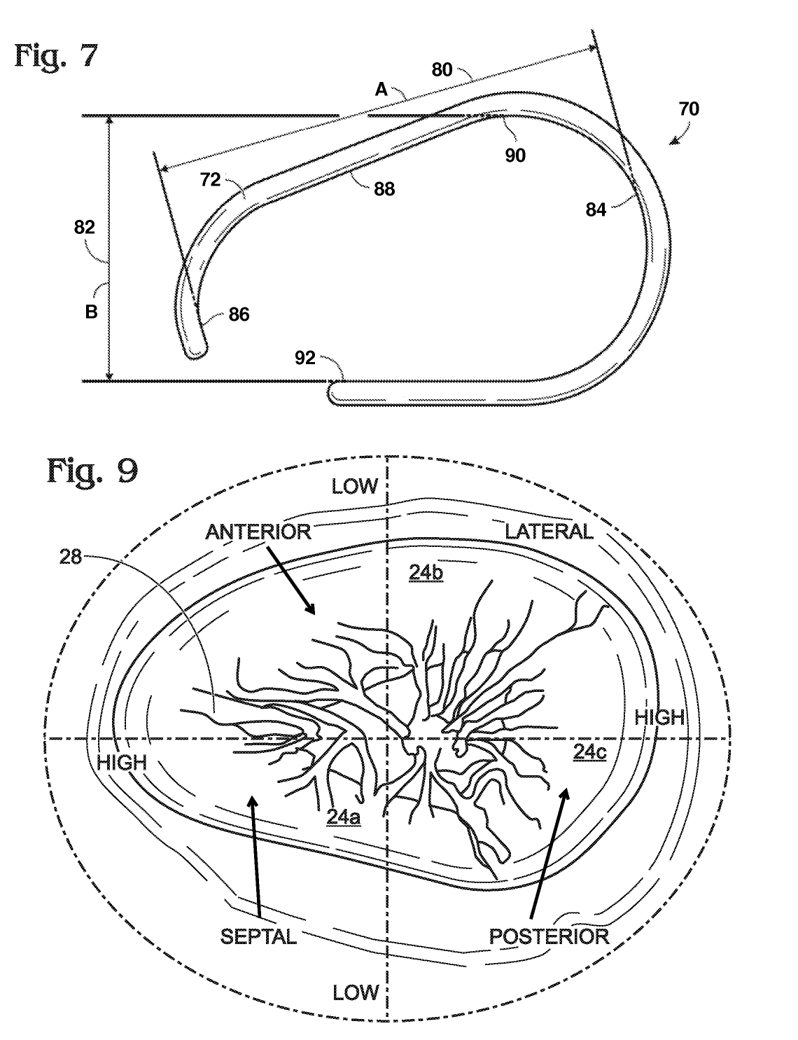

One embodiment of a tricuspid ring according to the present disclosure is shown in plan view in FIG. 7. Tricuspid ring 70 can comprise a ring 72 and subvalvular device (not shown) that mimics the shape of the native valve and right ventricle. The tricuspid ring 70 can thus at least partially restore the correct anatomy of a tricuspid valve annulus and right ventricle into which the ring 70 is implanted. Suitable subvalvular devices are described in U.S. Patent Publication No. 2010/0063586 to Hasenkam, which is incorporated herein by reference, in its entirety.

For instance, a ring and subvalvular system according to one embodiment of the present application includes a tricuspid annuloplasty ring 70 and a tension and anchoring subsystem adapted to align the papillary muscles with the tricuspid annulus, and to align the wall of the right ventricle with respect to the tricuspid valve in order to eliminate regurgitation. The tension and anchoring subsystem comprises a set of tension members, e.g. in the form of strings or sutures. Each of the tension members comprises a first end routed through the tricuspid ring 70 to a position at the exterior of the heart for adjustment of a set of anatomical lengths/distances defining the geometry of the right ventricle of the heart. Second ends fix to a position on or through the papillary muscles. The tricuspid ring 70 in this embodiment is either hollow to allow passage of the tension members, or otherwise includes channels that route the tension members. The tricuspid ring 70 attaches to the annulus, and its rigidity will support the geometry of the annulus via the tension members once they are fixed to the ring. Preferably, one or more tension members extend from one side of the tricuspid ring 70 and one or more tension members extend from the opposite side.

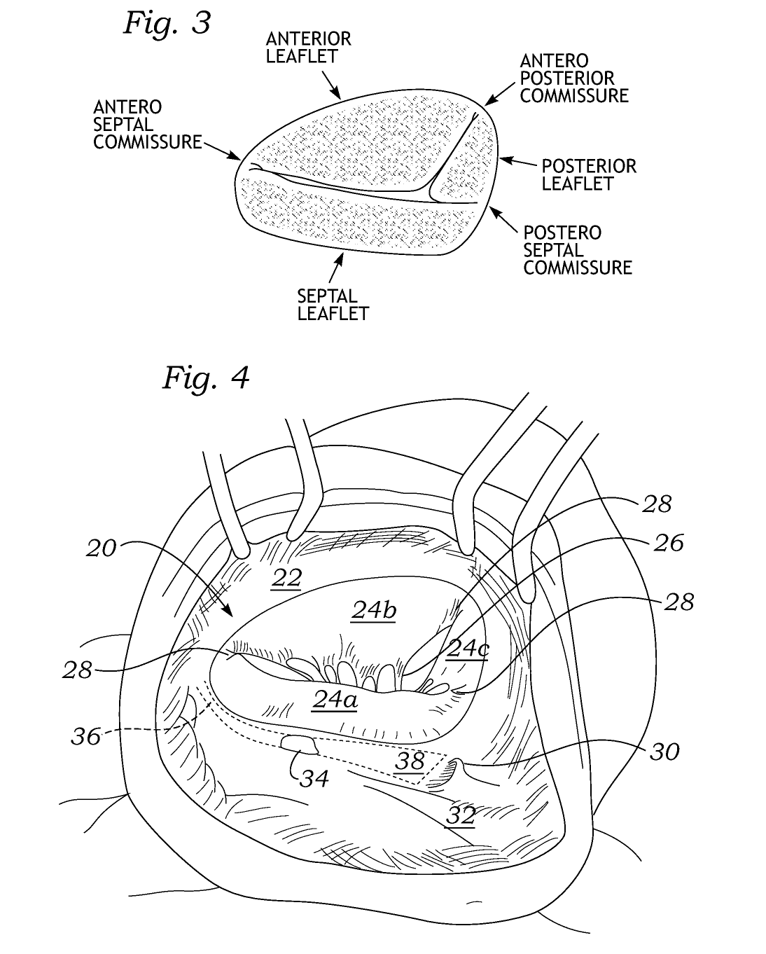

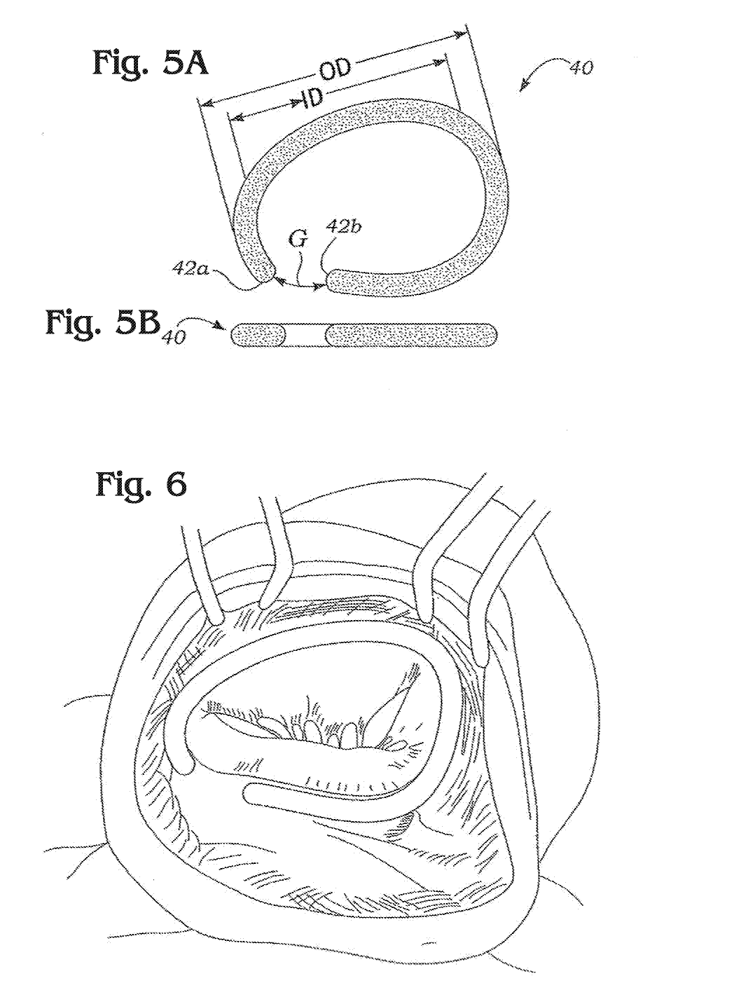

Tricuspid annuloplasty rings 70 disclosed herein can at least partially restore the anatomically correct shape in all three dimensions. As seen in FIG. 7, the shape of a tricuspid ring 70 is asymmetric and generally ovoid surrounding an axis in the direction of blood flow through the ring, and can be partially defined or characterized by a major axis 80 along its length and a minor axis 82 along its width, and more specifically, by the ratio of the major axis 80 to the minor axis 82. In terms of anatomical references, the length dimension of the tricuspid ring 70 when implanted extends generally from the middle of the posterior leaflet to the antero-septal commissure, as seen in FIG. 3, while the width dimension extends generally from the anterior leaflet adjacent the antero-posterior commissure to the septal leaflet. The major axis 80 is defined by the length A between a first point 84 and a second point 86 located on the interior 88 of the tricuspid ring 70. The length A represents the length of the line spanning the greatest length between two points on the interior 88 of the ring 70. The minor axis 82 is defined by the vertical displacement B between a third point 90 and a fourth point 92 on the interior 88 of the tricuspid ring 70. The length B represents the length of the line spanning the greatest width between two points on the interior 88 of the ring 70. Prior art tricuspid rings disclose designs having a major to minor axis ratio of 1.55. Tricuspid rings according to the present disclosure can be designed to have a major to minor axis ratio greater than that of prior art tricuspid rings. For example, the ratio can be around 1.56 or greater, such as between about 1.56 and about 2. Increasing the major to minor axis ratio can reduce residual tricuspid regurgitation post-operatively in some embodiments, such as by increasing septal-posterior coaptation.

The tricuspid rings of the present disclosure can be designed and manufactured in several different sizes, to form a set of tricuspid rings of various sizes. For example, a set of tricuspid rings can include ring sizes ranging from 24 mm to 40 mm, at intervals of 2 mm. Once again, the “ring size” is the size labeled on the particular annuloplasty ring packaging. A “set of rings” means a collection of annuloplasty rings of different sizes marketed together as one type of ring or for the same pathological condition, typically under one tradename. Although a set of rings is made available by the manufacturer, customers such as hospitals regularly order one or two sizes as needed, though orders of multiple sizes and even whole sets occur to maintain a supply of different sized rings on site. Smaller and larger sizes of rings can also be included in sets of tricuspid rings. In some embodiments of a set of tricuspid rings, the major to minor axis ratios can be the same for each size ring in the set. In other embodiments of a set of tricuspid rings, the major to minor axis ratios can vary for each different size of tricuspid ring. For example, in some embodiments, the major to minor axis ratio can increase with decreasing ring size. Thus, within a set of tricuspid rings, the major to minor axis ratio of one size of ring can be greater than the major to minor axis ratio of the next smaller sized ring. In some embodiments, the major to minor axis ratio can decrease with increasing ring size. Thus, within a set of tricuspid rings, the major to minor axis ratio of one size of ring can be less than the major to minor axis ratio of the next larger sized ring. As a result of the varying major to minor axis ratios, the minor axis 82 can more aggressively decrease in length in smaller sizes of tricuspid rings.

Incidence of tricuspid regurgitation can be further reduced by selecting a tricuspid ring size smaller than would conventionally be selected for a particular subject.

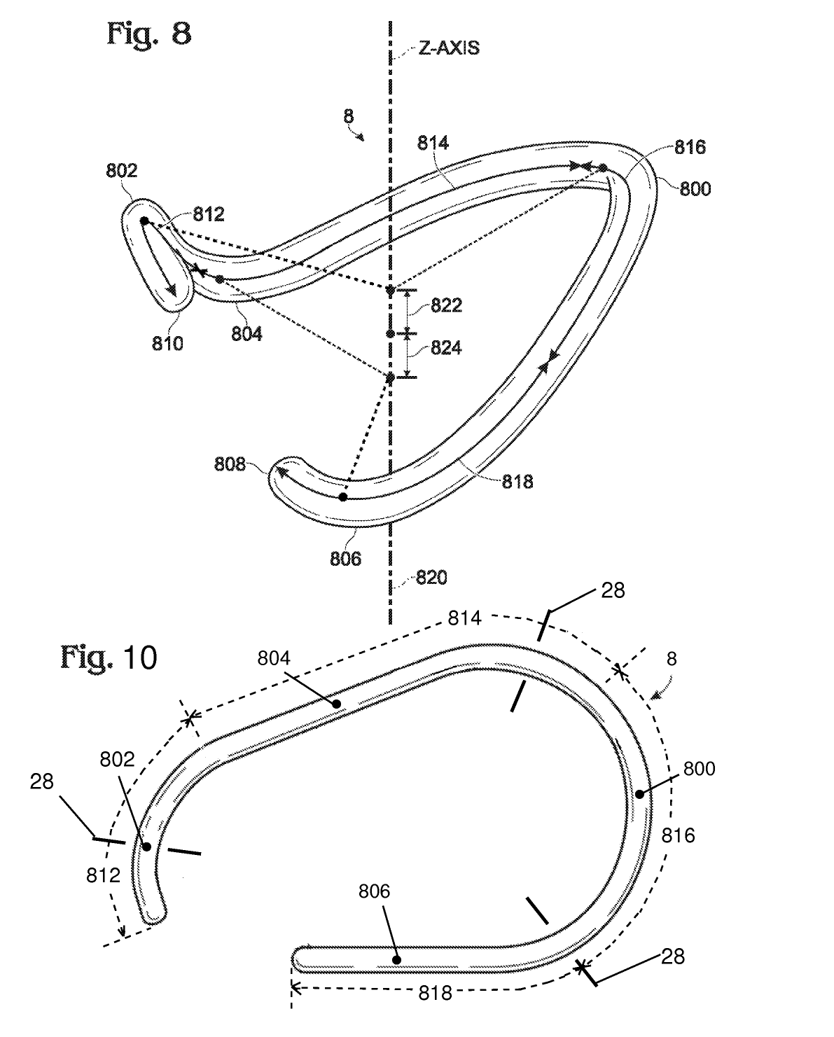

Furthermore, as seen in FIG. 8, embodiments of a tricuspid ring can be designed to substantially restore the anatomically correct shape to the valve annulus and/or right ventricle along the Z axis 820. The anatomically correct valve annulus includes two local high points (indicated by HIGH in FIG. 9), and two local low points (indicated by LOW in FIG. 9), along the Z axis, thus forming a bimodal saddle shape, as seen in FIG. 8. A tricuspid ring can be designed to account for the elevation of the native annulus' high and low points, and thus help correct the shape of a diseased annulus along the Z axis.

Embodiments of a tricuspid ring according to the present disclosure can include one or more points or portions of elevation in the Z direction, such as a primary saddle and a secondary saddle. As used herein, the elevation of a point refers to the distance of that point from the X-Y plane bisecting the tricuspid ring (i.e., the distance along the Z axis from a plane perpendicular to the blood flow through the ring that passes through the center of the overall elevation span of the ring). The static elevation of a point refers to the elevation of that point while the tricuspid ring is static and not implanted. When the tricuspid ring is implanted in a native valve, the elevation of some points can change with each cardiac cycle. The elevation of a portion or segment of a tricuspid ring refers to the elevation of the highest and lowest points of that portion or segment. The amplitude of the tricuspid ring is defined as the distance along the Z axis between a high point (e.g., the highest high point or a local maximum point) and a low point (e.g., the lowest low point or a local minimum point) of the ring. Thus, the amplitude can be determined by summing the absolute value of the elevations of the high and low points of the ring. An amplitude of a portion or segment of the tricuspid ring is defined by the distance along the Z axis between the highest point of that segment above the X-Y plane and the lowest point of that segment below the X-Y plane.

Portions of the elevated segments of the ring can correspond to native valve anatomy. For example, a tricuspid ring can include a primary saddle located at the posterior leaflet of the native valve when implanted in the valve annulus, with the lowest point of the primary saddle, for example, within the anterior leaflet. The elevation of the primary saddle can be about 2 mm in the Z direction. A high point of a secondary saddle can be located at the antero-septal commissure of the native valve when implanted in the valve annulus, and can have an elevation of about 0.5 mm.

In one embodiment of a tricuspid ring seen in FIGS. 8 and 10, the ring 8 can have high points 800, 802 at approximately the center of the posterior leaflet and at approximately the antero-septal commissure (the aortic bulge), respectively, when implanted. The elevation of the antero-septal commissure can be from about 0.5 mm to about 4 mm, and the elevation of the center of the posterior leaflet can be from about 2 mm to about 4 mm. For example, the local high point 800 can be a vertical distance 822 along the Z axis 820 above an X-Y plane cutting through the center of the ring 8. Embodiments of a tricuspid ring 8 can have low points 804, 806 at approximately the lateral center of the anterior leaflet and at approximately the center of the septal leaflet, when implanted. The elevation of the center of the anterior leaflet can be from about −2 mm to about −4 mm, and the elevation of the center of the posterior leaflet can be from about −1 mm to about −4 mm. For example, the local low point 804 can be a vertical distance 824 along the Z axis 820 below an X-Y plane cutting through the center of the ring 8.

FIG. 10 shows the tricuspid annuloplasty ring 8 in plan view, with segments (812, 814, 816, 818) and saddle points (800, 802, 804, 806) corresponding to FIG. 8. For reference to the native anatomy, the approximate location of the three commissures 28 as depicted in FIGS. 3 and 9 are indicated.

FIG. 9 illustrates reference anatomy that corresponds to high points and low points of a tricuspid ring when implanted. FIG. 9 shows the approximate locations of the local maxima, or high points, (indicated by HIGH) in the native valve, at about the center of the posterior leaflet 24c and at approximately the antero-septal commissure 28. FIG. 9 also shows the approximate locations of the local minima, or low points, (indicated by LOW) in the native valve, at about the center of the anterior leaflet 24b and at about the center of the septal leaflet 24a.

Further, some areas of a tricuspid ring can have a greater positive elevation than others. For example, as seen in FIG. 8, a lateral-posterior segment 816 can have a greater elevation than a septal-anterior segment 812. For example, in some embodiments, the elevation at the septal-anterior segment 812 can be between about 0.5 mm and about 10 mm, or between about 0.5 mm and about 6 mm. In some embodiments, the elevation at the lateral-posterior segment 816 can be between about 2 mm and 10 mm, or between about 2 mm and 6 mm.

In some embodiments, an anterior-lateral segment 814 can have a greater (e.g., more pronounced) negative elevation than a posterior-septal 818 segment. For example, in some embodiments, the elevation at the anterior-lateral segment 814 can be between about 2 mm and about 10 mm, or between about 2 mm and about 6 mm. In some embodiments, the elevation at the posterior-septal segment 818 can be between about 1 mm and 10 mm, or between about 1 mm and 6 mm.

In some embodiments, the total height, or the maximum distance between the highest point of the tricuspid ring 8 along the Z axis 820 and the lowest point of the tricuspid ring 8 along the Z axis 820 is about 20 mm or less (e.g., a total amplitude of about 10 or 15 mm), as measured from the center of the ring 8 at the highest point to the center of the ring 8 at the lower point, along the Z axis. In some embodiments, the height along the Z axis 820 of the tricuspid ring 8 is about 15% of the width of the tricuspid ring (e.g., the major axis length A, as seen in FIG. 7). For example, the height of a tricuspid ring can be about 5 mm for a 36 mm ring.

Sizing a tricuspid ring as described can yield advantages in some embodiments, such as producing a tricuspid ring that more accurately mimics the shape of the native tricuspid valve, imparting less stress on the valve tissues and annulus, and improving short and long term outcomes for treating tricuspid regurgitation and other abnormalities in the tricuspid valve.

In some embodiments of a set of tricuspid rings, the proportional elevation in the Z direction can remain substantially constant as the size of the ring increases. For example, each tricuspid ring in a set of rings can have a ratio of elevation in the Z direction to the width A within the range of from about 15% to about 25%. In some embodiments of a set of tricuspid rings, the proportional elevation in the Z direction can increase or decrease as the size of the ring increases. For example, the elevation can increase in proportion to the increasing major axis dimension A, such as increasing from about 15% to about 25%, or decrease in proportion to the increasing major axis dimension A, such as decreasing from about 25% to about 15%, as the size of the ring increases.

There are several reasons for varying the proportional elevation to width for different ring sizes. For example, for subjects with severe cases of tricuspid regurgitation and/or severe damage to the right ventricle, it can be advantageous to provide a progressively decreasing height to width ratio, such as a height to width ratio that decreases progressively from about 25% to about 5% over a size range of 24 mm to 40 mm rings. This could mean, for instance, that the absolute elevations around the ring remain the same as the ring size increases, or that the elevations increase but at a slower rate than the major and minor axes. The tissue of the tricuspid annulus is somewhat more fragile than other valve annuli such as the mitral valve, and proportionally raising or lowering segments of the ring may place excessive stress on the tissue during the cycling motion of the annulus. Thus, a set of similarly contoured rings whose major and minor axes increase but whose elevations remain substantially constant, or increase at a lower rate than the ring size, help reduce the chance of damaging the fragile annulus tissue.

Embodiments of a tricuspid ring can be configured to mimic the motion of a native tricuspid valve during the cardiac cycle, and can thereby substantially or at least partially restore the anatomically correct motion of the tricuspid valve annulus in the X-Y plane and/or the Z direction.

The orifice of disclosed tricuspid rings can expand during diastole and contract during systole, such that the area of the orifice expands from about 20% to about 40% during diastole. In one specific embodiment, the area of the orifice can expand an average of about 29% during a series of cardiac cycles. The orifice of disclosed tricuspid rings can expand an amount sufficient to allow efficient filling of the ventricle during diastole. At a later point in each cardiac cycle, the orifice of disclosed tricuspid rings can contract an amount sufficient to provide an efficient sphincter-like motion to substantially effectively seal the repaired valve shut during the increased ventricular pressure of systole.

Expansion and contraction of the orifice area and circumference of disclosed tricuspid rings can be accomplished in any suitable fashion. In some exemplary embodiments, such expansion and contraction can be provided by mechanisms such as one or more springs, polymeric materials, and/or an accordion-like core construction.

Similarly, the diameter (e.g., the major axis A and/or the minor axis B) of the tricuspid ring can expand and contract during the cardiac cycle. In some embodiments, the diameter of the tricuspid ring can increase by a percentage of from about 14.7% to about 17.2%. In one specific embodiment, the diameter of the tricuspid ring expands by about 16% during diastole. In some embodiments, the orifice expansion and the diameter increase is not evenly distributed around the circumference of the ring. For example, some embodiments of a tricuspid ring according to the present disclosure avoid expansion at the commissures. Such an arrangement can substantially prevent or reduce leakage through commissural clefts after implantation. On the other hand, segments of disclosed tricuspid rings corresponding to the center of each of the three native valve leaflets can be configured to expand.

Expansion and contraction of the diameter of disclosed embodiments of a tricuspid ring can be provided by any suitable fashion. For example, tricuspid rings according to the present disclosure can be provided with mechanisms such as springs, polymeric materials, an accordion-like core construction, selectively segmented core sections, selectively flexible core materials, one or more hinge points creating a jaw-like expansion, and/or a cable-based core design. For example, U.S. Patent Publication No. 2009/0287303 to Carpentier, which is incorporated by reference, describes various constructions of a tricuspid ring that can be incorporated in the embodiments disclosed in the present disclosure.

In some embodiments of sets of tricuspid rings, different sizes of tricuspid rings can be configured to expand to a greater or lesser extent during the cardiac cycle. For example, in some embodiments of a set of tricuspid rings, the larger size rings can be configured to undergo a larger orifice area expansion and/or a greater diameter increase than the small size rings.

Similarly, embodiments of a tricuspid ring can be configured for desirable movement in the Z direction, in order to at least partially restore anatomically correct movement of the native valve. For example, the elevation of embodiments of a tricuspid ring can increase during the systolic heart contraction and decrease during diastolic filling. Such movement can decrease leaflet stress during systole and/or decrease stress on the annuloplasty sutures holding the ring in place, which can reduce incidence of dehiscence.

The change in the elevation of the tricuspid ring can coincide with a change in circumference of the ring. For example, an increase in the elevation of the ring in the Z direction can coincide with a decrease in the circumference of the ring. Such movement can increase efficiency in opening and closing of the tricuspid valve.

Further, in embodiments of a set of tricuspid rings, the movement, or change in amplitude, in the Z direction can vary according to the size of tricuspid ring. For example, larger sizes of rings can be configured to undergo a relatively larger change in amplitude (e.g., a larger increase in elevation). Thus, the movement of the tricuspid ring in the Z direction can increase with increasing ring size.

In some embodiments of a tricuspid ring, the ring can comprise a plurality of segments. The term “segments” can refer different areas or portions along a continuous ring body. In such embodiments, different segments of the ring can be configured to different amplitude changes in the Z direction during the cardiac cycle. For example, still with reference to FIG. 8, the elevation of the septal-anterior segment 812 can decrease by approximately 1 mm. In some embodiments, the elevation can change by between about 0 mm and about −2 mm (e.g., move about 0 to 2 mm down in the Z direction, below the X-Y plane). The elevation of the anterior-lateral segment 814 can substantially remain unchanged during the cardiac cycle in some embodiments. The elevation of the lateral-posterior segment 816 can increase by approximately 1 mm, or between about 1 mm and about 2 mm. The elevation of the posterior-septal segment 818 can decrease by approximately 1 mm, or between about 0 mm and about −2 mm. In some embodiments, the elevation increase of the lateral-posterior segment 816 is the largest movement seen in the ring circumference. The lateral-posterior segment 816 of the tricuspid ring 8 can be associated with the lateral free wall of the right ventricle when implanted.

The incomplete, C-shaped tricuspid ring therefore experiences an out-of-plane motion of the free ends 808, 810 of the ring 8 with the septal-anterior free end 810 decreasing in the vertical axis and the posterior-septal free end 808 increasing in the vertical axis. The result is that the free ends 808, 810 of the ring move separately from each other with the distance between the two increasing by at least about 1 mm and by as much as about 4 mm. In some embodiments, the static vertical distance (along the Z axis) between the two free ends 808, 810 is between about 0 mm and about 6 mm. Thus, the total vertical distance between the two free ends 808, 810 in a dynamic heart with a dynamic ring (e.g., a ring that undergoes movement in the Z direction during the cardiac cycle) is between about 0 mm and about 10 mm.

Embodiments of tricuspid rings can provide for movement in the Z direction by any suitable design features. For example, some embodiments comprise specifically designed ring cores that include polymeric materials with varying flexibilities, stacked Elgiloy core members, a ring core that is thinner in height (along the Z axis) than in thickness (along the X-Y plane), and/or a composite core design, such as a metallic and polymer composite core design.

Some embodiments of a tricuspid ring can have a flexibility that varies along the length of the ring, such as having a relatively stiff first segment and getting progressively more flexible to a relatively flexible fourth segment. This varying flexibility can allow the ring to adapt (harmonize) its motion and three-dimensional shape to that of the annulus, rather than impose its own motion and 3-D geometry thereto which tends to increase the risk of ring dehiscence. In particular, the motion of the tricuspid annulus during systole-diastole is believed to exert some torsional forces on the implanted ring, and the variable flexibility accommodates such torques. Localized points of flexibility or “hinges” around the ring can conform and harmonize the physical properties of the ring to the annulus motion, while at the same time providing the needed corrective support.

Embodiments of a tricuspid ring can comprise an inner core encompassed by an elastomeric interface and an outer fabric covering. The inner core can extend substantially around the entire periphery of the ring body and can be a material such as stainless steel, titanium, Elgiloy (an alloy primarily including Ni, Co, and Cr), and/or polymers. Any material suitable to support the annulus while allowing for the movement described above can be used.

More specifically, the inner core is formed from a relatively rigid material such as stainless steel, titanium, and Cobalt Chromium (CoCr family of alloys: CoCr, L605, MP, MP25, MP35N, Elgiloy, FW-1058). The term “relatively rigid” refers to the ability of the core to support the annulus without substantial deformation, and implies a minimum elastic strength that enables the ring to maintain its original shape after implant even though it may flex somewhat. Indeed, as will be apparent, the ring desirably possesses some flexibility around its periphery. To further elaborate, the core would not be made of silicone, which easily deforms to the shape of the annulus and therefore will not necessarily maintain its original shape upon implant. Instead, the ring core is preferably formed from one of the relatively rigid metals or alloys listed above, or even a polymer that exhibits similar material and mechanical properties. For instance, certain blends of Polyether ether ketone (PEEK) with carbon and an alloy might be used, in which case the core could be injection molded.

In some embodiments, the elastomeric interface can be silicone rubber molded around the core, or a similar expedient. The elastomeric interface can provide bulk to the ring for ease of handling and implant, and can permit passage of sutures. The fabric covering can be any biocompatible material such as, for example, Dacron® (polyethylene terepthalate).

Disclosed tricuspid rings can possess a varying flexibility around its periphery. For example, the ring can be stiffer adjacent the first free end than adjacent the second free end, and can have a gradually changing degree of flexibility for at least a portion in between. For instance, the first segment can be relatively stiff while the remainder of the ring body gradually becomes more flexible through the second segment, third segment, and fourth segment.

It should also be understood that features of the present tricuspid ring can also be applicable and beneficial to rings for other of the heart's annuluses, such as the mitral valve annulus.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

What is claimed is:

1. A tricuspid annuloplasty ring for use in a tricuspid valve repair procedure, the tricuspid annulus having peripheral landmarks as viewed from above in a clockwise direction of an antero-septal commissure, anterior leaflet, posterior commissure, posterior leaflet, postero-septal commissure, and septal leaflet, comprising a core made of a relatively rigid material defined by:
a septal-anterior segment located around portions of the septal and anterior leaflets when implanted having a free first end and a second end;
an anterior-lateral segment located around portions of the anterior and posterior leaflets when implanted having a second end and a first end adjacent the second end of the septal-anterior segment;
a lateral-posterior segment located around the posterior leaflet when implanted having a second end and a first end adjacent the second end of the anterior-lateral segment; and
a posterior-septal segment located around the septal leaflet when implanted having a free second end and a first end adjacent the second end of the lateral-posterior segment,
wherein the ring is configured such that a gap exists between the free first end of the septal-anterior segment and the free second end of the posterior-septal segment, the ring having a bimodal saddle shape having a first and second high point and a first and second low point, the first high point being located within the septal-anterior segment, the second high point being located within the lateral-posterior segment, the first low point being located within the anterior-lateral segment, and the second low point being located within the posterior-septal segment.

2. The tricuspid annuloplasty ring according to claim 1, wherein the ratio of the greatest length between any two points on an interior surface of the ring to the greatest width between any two points on the interior of the ring is at least 1.56.

3. The tricuspid annuloplasty ring according to claim 1, further comprising a subvalvular apparatus.

4. The tricuspid annuloplasty ring according to claim 1, wherein the ring is configured to substantially restore the anatomically correct shape in all three dimensions of a native tricuspid valve in which the ring is designed to be implanted.

5. The tricuspid annuloplasty ring according to claim 1, wherein when the ring is positioned within a native tricuspid valve, the first high point of the ring is approximately positioned adjacent the septal-anterior commissure of the native tricuspid valve and the second high point of the ring is approximately positioned adjacent the center of the posterior leaflet of the native tricuspid valve.

6. The tricuspid annuloplasty ring according to claim 5, wherein the elevation of the first high point is from about 0.5 mm to about 4 mm.

7. The tricuspid annuloplasty ring according to claim 5, wherein the elevation of the second high point is from about 2 mm to about 4 mm.

8. The tricuspid annuloplasty ring according to claim 1, wherein when the ring is positioned within a native tricuspid valve, the first low point of the ring is approximately positioned adjacent the center of the anterior leaflet of the native tricuspid valve and the second low point of the ring is approximately positioned adjacent the center of the septal leaflet of the native tricuspid valve.

9. The tricuspid annuloplasty ring according to claim 8, wherein the elevation of the first low point is from about −2 mm to about −4 mm.

10. The tricuspid annuloplasty ring according to claim 8, wherein the elevation of the second low point is from about −1 mm to about −4 mm.

11. The tricuspid annuloplasty ring according to claim 1, wherein the ring is configured to move during the normal cardiac cycle once implanted in a native valve, such that a first elevation of one or more of the high points and a second elevation of one or more of the low points change during each cardiac cycle.

12. The tricuspid annuloplasty ring according to claim 1, wherein the ring is configured to move during the normal cardiac cycle once implanted in a native valve, such that the diameter of the ring changes during each cardiac cycle.

13. The tricuspid annuloplasty ring according to claim 1, wherein the ring is configured to move during the normal cardiac cycle once implanted in a native valve, such that the area of an orifice defined by the ring changes during each cardiac cycle.

14. A set of a plurality of tricuspid annuloplasty rings of different sizes, each ring being adapted for use in a tricuspid valve repair procedure, the tricuspid annulus having peripheral landmarks as viewed from above in a clockwise direction of an antero-septal commissure, anterior leaflet, posterior commissure, posterior leaflet, postero-septal commissure, and septal leaflet, wherein each ring comprises a core made of a relatively rigid material defined by:
a septal-anterior segment located around portions of the septal and anterior leaflets when implanted having a free first end and a second end;
an anterior-lateral segment located around portions of the anterior and posterior leaflets when implanted having a second end and a first end adjacent the second end of the septal-anterior segment;
a lateral-posterior segment located around the posterior leaflet when implanted having a second end and a first end adjacent the second end of the anterior-lateral segment; and
a posterior-septal segment located around the septal leaflet when implanted having a free second end and a first end adjacent the second end of the lateral-posterior segment,
wherein the ring is configured such that a gap exists between the free first end of the septal-anterior segment and the free second end of the posterior-septal segment, the ring having a bimodal saddle shape having a first and second high point and a first and second low point, the first high point being located within the septal-anterior segment, the second high point being located within the lateral-posterior segment, the first low point being located within the anterior-lateral segment, and the second low point being located within the posterior-septal segment.

15. The set of tricuspid annuloplasty rings according to claim 14, wherein each ring has a ring ratio of the greatest length between any two points on an interior surface of the ring to the greatest width between any two points on the interior of the ring, and wherein the ratio is different for each ring in the set.

16. The set of tricuspid annuloplasty rings according to claim 15, wherein when the set of rings is ordered from the smallest ring to the largest ring, the change in the ring ratio from one ring to the next largest ring is not constant.

17. The set of tricuspid annuloplasty rings according to claim 14, wherein an elevation of the first and second high points varies with each different sized ring in the set.

18. The set of tricuspid annuloplasty rings according to claim 17, wherein each ring is configured to move during the normal cardiac cycle when implanted in an native valve such that the elevation of the first and second high points changes during each cardiac cycle, and wherein each ring is configured to undergo a larger change in the elevation of the first and second high points than the next smaller ring in the set.

19. The set of tricuspid annuloplasty rings according to claim 14, wherein the elevation of the first and second low points varies with each different sized ring in the set.

20. The set of tricuspid annuloplasty rings according to claim 19, wherein each ring is configured to move during the normal cardiac cycle when implanted in an native valve such that the elevation of the first and second low points changes during each cardiac cycle, and wherein each ring is configured to undergo a larger change in the elevation of the first and second low points than the next smaller ring in the set.

21. A tricuspid annuloplasty ring for use in a tricuspid valve repair procedure, the tricuspid annulus having peripheral landmarks as viewed from above in a clockwise direction of an antero-septal commissure, anterior leaflet, posterior commissure, posterior leaflet, postero-septal commissure, and septal leaflet, comprising a core made of a relatively rigid material defined by:
a septal-anterior segment located around portions of the septal and anterior leaflets when implanted having a free first end and a second end;
an anterior-lateral segment located around portions of the anterior and posterior leaflets when implanted having a second end and a first end adjacent the second end of the septal-anterior segment;
a lateral-posterior segment located around the posterior leaflet when implanted having a second end and a first end adjacent the second end of the anterior-lateral segment; and
a posterior-septal segment located around the septal leaflet when implanted having a free second end and a first end adjacent the second end of the lateral-posterior segment,
wherein the ring is configured such that a gap exists between the free first end of the septal-anterior segment and the free second end of the posterior-septal segment, the ring having an undulating contour with a local high point located within the septal-anterior segment at the antero-septal commissure when implanted, and a local low point located within the posterior-septal segment.

22. The tricuspid annuloplasty ring according to claim 5, wherein the elevation of the local high point is from about 0.5 mm to about 4 mm.

23. The tricuspid annuloplasty ring according to claim 5, further including a second local high point located within the lateral-posterior segment and having an elevation of from about 2 mm to about 4 mm.

24. The tricuspid annuloplasty ring according to claim 8, wherein the elevation of the local low point is from about −2 mm to about −4 mm.

25. The tricuspid annuloplasty ring according to claim 8, further including a second local low point located within the posterior-septal segment and having an elevation of from about −1 mm to about −4 mm.
